(19) United States
(12) Patent Application Publication
(10) Pub. No.: US 2023/0363790 A1
(43) Pub. Date: Nov. 16, 2023

(54) GONIOTOMY DEVICES WITH FLUID INGRESS/EGRESS CONTROL

(71) Applicant: MICROSURGICAL TECHNOLOGY, INC., Redmond, WA (US)

(72) Inventors: Haley HUNT, Issaquah, WA (US); Dave SCOTT, Bothell, WA (US); John GARLAND, Coto de Caza, CA (US)

(73) Assignee: MICROSURGICAL TECHNOLOGY, INC., Redmond, WA (US)

(21) Appl. No.: 18/026,996

(22) PCT Filed: Sep. 17, 2021
(86) PCT No.: PCT/US2021/050793

§ 371 Date: Mar. 17, 2023

Related U.S. Application Data
(60) Provisional application No. 63/080,779, filed on Sep. 20, 2020.

(51) Int. Cl. A61B 17/34; A61F 9/007

(52) U.S. Cl. CPC A61B 17/3421; A61F 9/00736; A61B 2017/3419; A61B 2217/007; A61B 2017/00862

(57) ABSTRACT

A wound sealing sleeve for use with a goniotomy device has a hollow body with an open end by which the sleeve is attachable to a handle and an open tip through which a shaft is passed. One or more optional ports are formed in the sleeve to provide variable fluid paths. The port or ports may differ in size, shape and positioning on the sleeve. The wound sealing sleeve may be affixed to a handle by threads, a friction fit, glue, or mechanically fastened. The wound sealing sleeve is able to be deformed slightly in order to match the shape or contour of the eye incision to prevent leakage between the exterior surface of the wound sealing sleeve and the surgical incision.

### RELATED APPLICATION

This application claims priority benefit to U.S. Provisional Application No. 63/080,779, filed Sep. 20, 2020, which is fully incorporated herein by reference for all purposes.

### FIELD OF THE INVENTION

The present invention relates generally to the fields of medicine and engineering and more particularly to surgical devices and methods for performing surgery.

### BACKGROUND

In ophthalmology, various surgical procedures are known in which instruments are used to perform surgical interventions in the eye interior. In invasive intraocular procedures, at least one opening is created in the outer membrane of the eye through which an instrument is inserted into the eye interior.

This opening, or wound site, is not pressure-tight and may result in leaking of the aqueous humor. Because a lowering of the intraocular pressure should be avoided, irrigation liquid or viscoelastic fluid is introduced into the eye via the operating instrument itself, or via a separate second instrument, in order to be able to continuously compensate for the loss of pressure during the operation. The instrument may also contain channels for aspiration in which to maintain equilibrium and remove fluid introduced.

One intraocular surgical procedure is phacoemulsification, which has become the standard of care for cataract removal. The phacoemulsification process generally employs a small incision typically of about 2 millimeters (mm) to about 4 mm in length through the cornea and a probe is used to ultrasonically break apart and remove the crystalline lens through the capsulorhexis. During the cataract surgical procedure and immediately after the procedure, it is important to maintain the intraocular pressure at a desired level. This is particularly important to a subset of cataract patients that also have glaucoma.

Many studies have demonstrated that combined goniotomy and cataract surgery showed a significantly greater reduction in intraocular pressure with favorable safety profiles in open-angle glaucoma patients. Goniotomy is often performed after cataract due to the common blood reflux during the removal of the trabecular meshwork and the possibility of hyphema. Goniotomy is a simple and directed technique of microsurgical dissection with mechanical disruption of the trabecular meshwork. In a surgical goniectomy, a tissue cutting or ablating device is inserted into the anterior chamber of the eye and used to remove a full thickness strip of the tissue from the trabecular meshwork overlying Schlemm's canal. The goniectomy procedure and certain prior art instruments useable to perform such procedure are described in WO2018/151808.

Cataract surgery requires a larger incision than goniotomy because of the implantation of the intraocular lens. Therefore, when goniotomy is performed on pseudophakic patients directly following cataract surgery, the surgical device is inserted into an incision that is significantly larger than the outer dimensions (up to about four times greater than the 1 mm device). This resultant gap created by the larger wound and small instrument may cause significant fluid egress, risking anterior chamber collapse if not managed. The egress is sometimes mitigated by partially stitching the incision before performing the goniotomy but it is not a flawless solution and is clinically disfavored. There still remains a need to prevent egress for a post-cataract goniotomy procedure.

Infusion sleeves used with phacoemulsification instruments to reduce the infusion fluid leakage from the incision wound are well-represented in the art and exemplify the attempts to address the problem of maintaining an adequate flow of irrigating liquid without causing damage to the eye.

U.S. Pat. No. 4,643,717 (Cook et al.) describes an aspiration fitting adapter formed as a sleeve concentric to the phaco needle and having a pair of bilaterally opposed discharge ports formed proximate the end of the sleeve to infuse irrigating liquid into the eye.

U.S. Pat. No. 5,084,009 (Mackool) discloses a surgical instrument for removing a cataract from a patient's eye including a hollow vibratable needle surrounded by a hollow infusion sleeve which conforms to the surgical incision and thereby prevents leakage from the incision, and also with means preventing the hollow infusion sleeve from collapsing against the hollow vibratable needle. The hollow infusion sleeve for use during eye surgery with the sleeve has a flattened cross-section and a pair of infusion ports formed on the forward portion of the flattened section.

U.S. Pat. No. 5,151,084 (Khek) describes an ultrasonic needle with an infusion sleeve that includes a baffle. The sleeve also fits concentrically about the needle and allows the needle to protrude a substantial distance therefrom while providing a pair of discharge ports bilaterally opposed to each other near the terminus of the sleeve.

U.S. Pat. No. 5,634,912 (Injev) discloses an infusion sleeve having a two-piece body. The first piece is conical in shape and is attached to the handpiece by conventional methods. The distal end of the first piece contains a series of annular grooves. The second piece is tubular in shape and contains an annular rim or lip on the proximal end that interlocks within the annular grooves on the first piece.

U.S. Pat. No. 5,879,356 (Geuder) describes a surgical instrument for crushing crystalline eye lenses by means of ultrasound and for removing lens debris by suction which demonstrates the use of a sleeve positioned concentric to the needle and having a pair of discharge ports formed thereon. The rinsing liquid is supplied to the site of surgery via a flow path extending between the hollow needle and the sleeve surrounding this needle with a spacing from the needle. This sleeve extends approximately along the entire length of the hollow needle and has at its distal end at least one outlet aperture for the rinsing liquid. The sleeve has two layers, in which an outer jacket layer is made of tissue compatible plastic such as silicone.

U.S. Pat. No. 6,007,555 (Devine) describes an ultrasonic needle for surgical emulsification and a clear silicon infusion sleeve having an infusion hole positioned proximal to the needle tip.

U.S. Pat. No. 6,117,151 (Urich et al.) discloses an eye incision temperature protection sleeve fitted concentrically about a needle and having a single discharge port through which irrigating liquid is passed. The sleeve has a flexible outer sleeve and a rigid inner sleeve that are both coupled to a case of the handpiece.

U.S. Pat. No. 6,605,054 (Rackley) describes a multiple bypass port phaco sleeve having multiple aspiration ports and a single discharge port to infuse liquid into the eye. The sleeve establishes an annular passage around the needle and enables irrigation fluid to pass into an eye through a cornea/sclera wound while cooling the needle.

U.S. Pat. No. 7,601,135 (Akahoshi) discloses an infusion sleeve for use with a phacoemulsification handpiece having a hollow body with an open end by which the sleeve is attachable to the handpiece and an open tip through which a phacoemulsification needle is passed. Irrigating liquid is directed from the handpiece through the sleeve. At least three discharge ports are formed in the sleeve to provide increased flow of irrigating liquid proximate the sleeve tip. The ports may differ in size, shape and positioning on the sleeve.

There remains a need in the art for a wound leakage management device that can be used collaboratively with a goniotomy surgical device to restrict fluid ingress or egress at the incision wound of the operating site while allowing for proper infusion and/or aspiration. Maintaining a sufficient amount of liquid and interior chamber pressure prevents collapse of certain tissues within the eye and attendant injury or damage to delicate eye structures. As a result, the goniotomy procedure would be significantly improved and clinically preferred if fluid leakage during intraocular surgery could be reduced or eliminated.

The need also exists for a wound leakage management device to be simple in construction, efficient in operation, and economical to manufacture.

### SUMMARY OF THE INVENTION

In view of the foregoing, an object of the present invention is to address the need for restricting fluid ingress or egress at the incision wound of the operating site by providing a wound sealing sleeve for use with a goniotomy device. It is also an object of the present invention to provide a wound sealing sleeve as a valvular device that allows for various fluid paths. A still further object of the present invention is to provide a goniotomy device with a variable capacity in the infusion line to allow multiple fluids to be transferred simultaneously through a small volume in micro-incisional glaucoma surgery.

Disclosed herein is a wound sealing sleeve for use with a goniotomy device. The wound sealing sleeve has a hollow body comprising a conical receiving area, a tubular passage and a tapering section. The conical receiving area has an open end by which the sleeve is attachable to a goniotomy device and the tapering section has an open tip through which a shaft of the device is passed. One or more optional ports or pathways are formed in the sleeve to provide additional source of fluidics transfer in or out of the eye. The port or ports may differ in size, shape and positioning on the sleeve. The wound sealing sleeve could be affixed to the device by threads, a friction fit, glue, or mechanically fastened.

In accordance with one aspect of the present invention, there is provided a handpiece goniotomy device with a wound sealing sleeve. The goniotomy device comprises a handle, a shaft, and a distal member or foot on a distal end of the shaft. The distal member or foot may have a forward tip, a right edge, a left edge and a transversely concave depression, cavity or space between the right and left edges. The distal member is insertable into Schlemm's canal of an eye and thereafter advanceable through Schlemm's canal such that trabecular meshwork tissue will be cut by the right and left edges. In some embodiments, the device may optionally include a fluidics control, lumens, opening or ports and associated connectors for infusing irrigation fluid and/or aspirating fluid and/or tissue or other debris from the eye.

In accordance with one embodiment, the handpiece goniotomy device is encased by a wound sealing sleeve. The wound sealing sleeve encases at least a portion of the fluidics control and a portion of the shaft and is spaced therefrom for fitting into the opening of the operating site, wherein the sleeve is configured to restrict fluid ingress or egress at the opening of the operating site.

The wound sealing sleeve allows glaucoma surgery to be performed using the wound access created from cataract surgery. The wound sealing sleeve is configured to seal and control fluid ingress or egress at the wound site. The wound sealing sleeve also enables pass through of instrumentation and allows for multiple interventions to access a single wound site, which significantly saves time and cost, and minimizes patient discomfort and engagement. The wound sealing sleeves of different sizes may be produced to accommodate a range of incision sizes. The use of the wound sealing sleeve does not interfere with visualization of the distal end effector

In view of the foregoing, other aspects, features, details, utilities, and advantages of the disclosed embodiments will be apparent from the following description and claims as well as the accompanying drawings.

### BRIEF DESCRIPTION OF THE DRAWINGS

The following detailed description and examples are provided for the purpose of non-exhaustively describing some, but not necessarily all, examples or embodiments of the invention, and shall not limit the scope of the invention in any way.

FIG. 1 shows one embodiment of a surgical handpiece device.

FIG. 2 is an enlarged view of a distal portion of the device of FIG. 1.

FIG. 3 is a side view of a distal portion of the device of FIG. 1.

FIG. 4 shows a top view of the wound sealing sleeve.

FIG. 5 shows a sectional view of the wound sealing sleeve shown in FIG. 4.

FIG. 6 illustrates that the surgical handpiece device is encased by a wound sealing sleeve.

### DETAILED DESCRIPTION OF THE EMBODIMENTS

It should be understood that this invention is not limited to the particular apparatus, methodology, protocols, and systems, etc., described herein and as such may vary. The terminology used herein is for the purpose of describing particular embodiments only and is not intended to limit the scope of the present invention, which is defined solely by the claims.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

The present disclosure provides apparatuses for goniotomy instruments that may be used in cataract treatment to restrict fluid egress at the wound incision site of an eye. To illustrate, several exemplary embodiments are described in detail herein. The apparatuses and methods described can be utilized in other contexts.

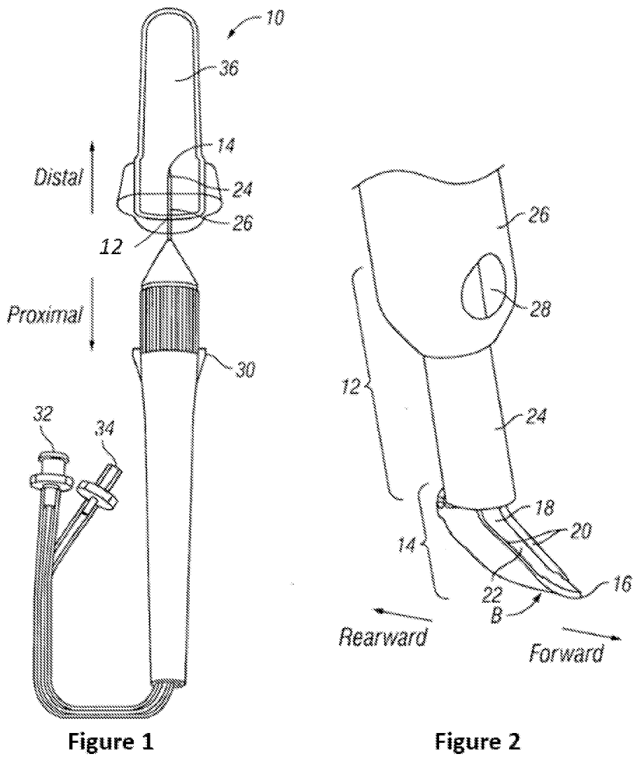

FIG. 1 depicts one embodiment of a goniotomy device as illustrated in WO2018/151808, the entirety of which is expressly incorporated herein by reference. This handpiece device 10 generally comprises a handle 30, a shaft 12 that extends distally from the handle 30 and a distal member 14 (which is alternatively referred to herein as a “foot”) on the distal end of the shaft 12. The shaft 12 comprises an inner tube 24 and an outer tube 26. The inner tube 24 extends out of and beyond the distal end of the outer tube 26.

Referring to FIG. 1, the device 10 may include lumens, outlets and connectors for infusion of irrigation fluid into and/or aspiration of fluid, tissue or other debris. In one embodiment, the device 10 includes tubular infusion and aspiration connectors 32, 34 for connecting sources of irrigation fluid and aspiration to the desired irrigation/aspiration lumens of the shaft 12. More specifically, in the particular non-limiting example shown, tubular connector 32 is configured for connection to a source of irrigation fluid and communicates with the annular lumen that extends through the outer tube around the outer surface of the inner tube 24, thereby facilitating infusion of irrigation fluid through the outer tube 26 and out of opening 28. Tubular connector 34 is configured for connection to an aspiration pump or suction source to facilitate aspiration of fluid, tissue or other debris, into the open distal end of inner tube 24, through the lumen of inner tube 24 and out of tubular connector 34.

As illustrated in FIG. 2, the shaft 12 comprises an inner tube 24 and an outer tube 26. The inner tube 24 extends out of and beyond the distal end of the outer tube 26. The inner tube 24 has a smaller outer diameter than an inner diameter of the outer tube 26 such that when inner tube 24 is positioned within the outer tube 26, for example, substantially coaxial therewith. The inner surface of the outer tube 26 is spaced apart from the outer surface of the inner tube 24 thereby defining an annular lumen which communicates with outlet port 28. The distal end of the outer tube 26 tapers down and is in sealing contact with the outer surface of the protruding inner tube 24. The opening 28 is positioned slightly above the location where the inner tube 24 exits the distal end of the outer tube 26.

Referring to FIG. 2, the distal member 14 has a bottom surface B, right and left upwardly extending side walls 22 and a cavity or open area 18 between the side walls 22 and rearward of the forward tip 16. The forward tip 16 may be tapered to a blunt point as shown. Edges 20 form the sides of the forward tip 16 and transition in orientation as they progress in the rearward direction to form spaced-apart, upwardly-sloping top surfaces of the sidewalls 22. All or portions of the upwardly sloping regions of edges 20 may be sharpened, beveled, serrated, or otherwise configured to form cutting regions 21 which facilitate cutting of tissue as it advances over those edges 20. Also, the side walls 22 and/or edges 20 may be non-parallel such that the space 18 between the sidewalls 22 and/or edges 20 becomes wider as it progresses in the rearward direction. This optional widening or non-parallelism of the side walls 22 and/or edges 20 may serve to transverse stretch or transversely tighten tissue as it advances up the progressively-widening, upwardly-sloping regions of edges 20.

In the non-limiting example shown, the upper surface of the distal member is generally trough-shaped (e.g., U-shaped), with the first and second inclined side walls 22 onto which are formed tissue-severing edges or blades 20. These edges 20 are spaced apart, as shown.

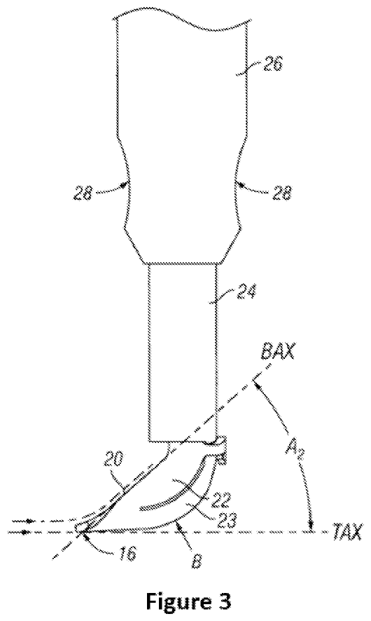

With specific reference to FIG. 3, as the distal member 14 advances through Schlemm's canal, trabecular meshwork (TM) tissue will ride over the edges 20 generally along a blade axis (BAX) as the edges incline. The angle A2 shown between transverse axis (TAX) and axis BAX depends on the angle of incline of each edge 20. The right edge 20R and left edge 20L may incline at the same or different angles A2. In the particular non-limiting example shown, both edges 20 incline at the same angle A2, which is preferably about 35 to about 40 degrees as shown in FIG. 3, as this will place the TM under tension over a reasonably short distance along path 20 of the member so that the length of the member can remain compact. However, it is reasonable to assume that angle A2 could provide good clinical utility over the range of about 30 to about 50 degrees, and maintain some clinical utility over a range between about 20 to about 70 degrees.

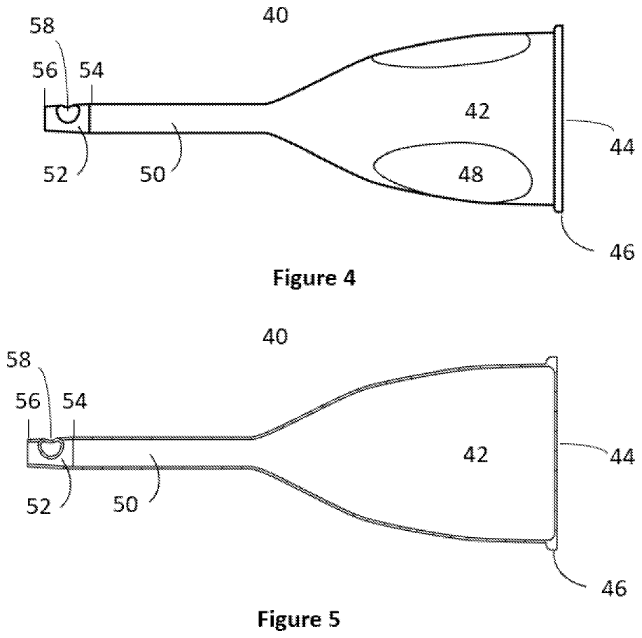

FIG. 4 shows a top view of the wound sealing sleeve 40 according to one embodiment of the present invention. FIG. 5 is a sectional view of the wound sealing sleeve 40 shown in FIG. 4. The wound sealing sleeve 40 comprises a receiving area 42 with a receiving space, which is conical in shape, for example, opens out at the proximal end 44 and is surrounded there by a fluidics control flange 46 that extends farther out than the rest of the wall of the receiving area 42. The receiving area 42 may further include one or more flats 48 in an oval shape or other shapes that could be used for finger grips. The receiving area 42 is connected to a tubular passage 50. The tubular passage 50 terminates at a tapering section 52 which serves as an insertion aid. A curved or tapered shoulder extends from the proximal end 54 of the tapering section 52 to the distal open end 56 of the sleeve 40. The distal open end 56 of the sleeve 40 is in sealing contact with the outer surface of the outer tube 26. One or more ports 58 are positioned on the tapering section 52. The depiction of the wound sealing sleeve 40 is for illustrative purposes, understanding that such sleeves are available in a number of different sizes and configurations.

The wound sealing sleeve may include a layer of a compliant biocompatible material, for example, a natural or synthetic rubber or elastomer and/or a thermoplastic elastomer. A particularly suitable material is silicone or silicone rubber (siloxane rubber) or ethylene propylene diene monomer (EPDM) elastomer. The wound sealing sleeve may be translucent to allow further visualization of the anterior chamber and confirmation of fluid transfer within the sleeve.

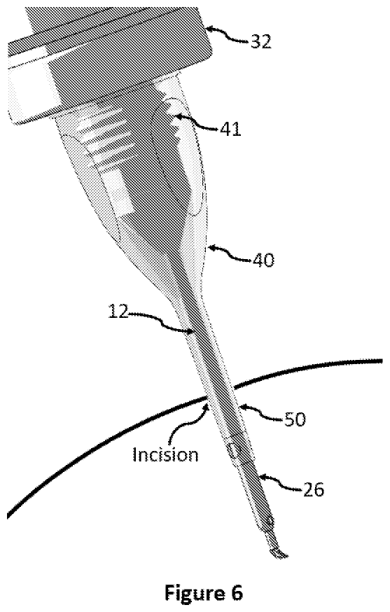

Referring to FIG. 6, the device 10 is now encased by a wound sealing sleeve 40. The end of the handle 30 and the fluidics control 32 of the device 10 are received in the receiving area 42 of the wound sealing sleeve 40. Threads 41 in the receiving area 42 are for this purpose turned onto an outer thread of the handle 30, such that a screwed union is obtained. It is contemplated that the wound sealing sleeve may be connected to the handle by a fluid tight connection, including threads, a friction fit, glue, or mechanically fastened. Preferably, the wound sealing sleeve may be disposable and rotationally or frictionally connected to the fluidics control 32 of the device 10. The wound sealing sleeve encases at least a portion of the fluidics control and a portion of the shaft and is spaced therefrom for fitting into the opening of the operating site.

The outer tube 26 of the device 10 extends through the tubular passage 50 of the wound sealing sleeve 40 and protrudes out of the wound sealing sleeve 40 and is thus available with its distal member 14 for the operation. The tubular passage 50 is radially spaced from the outer tube 26. The tubular passage 50 extends approximately about three-quarters of the entire length of the outer tube 26 of the device 10.

When the device is used, the shaft 12 of the device 10 encased by a wound sealing sleeve 40 is inserted to the eye through a small (about 2-4 mm) incision at the edge of the cornea into the anterior chamber of the eye which is to undergo the surgery. The diameter of the tubular passage 50 substantially matches the width of the incision so that a seal is formed by means of the exterior surface of the tubular passage 50 being engaged by and compressed by the edge of the incision. As a result, there is minimal leakage between the exterior surface of the wound sealing sleeve and the surgical incision. Wound sealing sleeves of a variable diameter tubular passage may be produced to accommodate a range of incision sizes (e.g., including about 2.2 to about 2.4 mm and about 2.6 to about 2.85 mm options). By being made of a relatively compressible layer, the sleeve of the present invention is able to be deformed slightly in order to match the shape or contour of the eye incision.

Referring to FIG. 6, it is contemplated that the handle 30 of the device 10 may have a fluidics control 32 which comprises multiple internal fluid channels (not shown) for delivering irrigation liquid through the port 58 of the sleeve 40. From an irrigation outlet into which the irrigation channel opens, the irrigation liquid then flows out of the handle 30 and through the space between the outer surface of the outer tube 26 and the interior wall of the wound sealing sleeve 40 surrounding the tubular passage 50, and then through the port 58 at the tapering section 52 of the wound sealing sleeve 40. It is further contemplated that one or more internal fluid channels of the fluidics control 32 are configured to facilitate aspiration of fluid, tissue or other debris from the eye through the port 58 and out of tubular connector 34.

In many procedures it will be beneficial to have active irrigation (to pressurize and maintain the volume of the anterior chamber and to open access between the top of iris and the inner surface of the cornea to facilitate access to the TM) and active aspiration (to clear visualization of the treatment area from debris and blood reflux) during angle surgery procedures. Active irrigation also generally eliminates the need to inject viscoelastics into the angle during surgery, saving surgical costs as well as improving the surgical view of the TM (use of viscoelastics often distorts the view of the TM due to changes in refractive index between the viscoelastics and the irrigation fluid). However, if the device 10 includes the optional infusion/aspiration capability as described herein and the user does not wish to use irrigation or aspiration in a particular procedure, the user may optionally purge the air from the I/A channels and connect the irrigation and aspiration connectors 32, 34 together. Alternatively, in some embodiments, the I/A channels may be plugged or non-existent. In procedures where irrigation and aspiration are not used or unavailable, viscoelastic may be injected into the anterior chamber of the eye to maintain access to and visualization of the angle during the procedure.

It is intended that all matter contained in the above description or shown in the accompanying drawings shall be interpreted as illustrative only and not limiting. Changes in detail or structure may be made without departing from the spirit of the disclosure as defined in the appended claims.

1. A goniotomy device comprising:
a handle;
a fluidics control;
a shaft having a distal portion that is insertable into an opening of an operating site of an eye, wherein the shaft comprises an outer tube and an inner tube disposed therein, wherein the inner tube extends through a lumen of the outer tube and a distal portion of the inner tube extends out of and beyond a distal end of the outer tube; and
a sleeve encasing at least a portion of the fluidics control and a portion of the shaft and spaced therefrom for fitting into the opening of the operating site, wherein the sleeve is configured to restrict fluid ingress or egress at the opening of the operating site.

2. The device of claim 1, wherein the sleeve comprises a conical receiving area, a tubular passage and a tapering section.

3. The device of claim 2, wherein the sleeve comprises one or more ports at the tapering section.

4. The device of claim 3, wherein the sleeve has a space between the shaft and the sleeve for receiving an irrigation fluid and for supplying the irrigation fluid through the port.

5. The device of claim 3, wherein the sleeve has a space between the shaft and the sleeve for aspirating fluid, tissue or other debris through the port.

6. The device of claim 2, wherein the sleeve comprises one or more flats in the conical receiving area for finger grips

7. The device of claim 1, wherein the sleeve is disposable.

8. The device of claim 1, wherein the sleeve is affixed to the device.

9. the device of claim 1, wherein the sleeve is affixed to the fluidics control by threads, a friction fit, glue, or mechanically fastened in a fluid tight manner.

10. The device of claim 1, wherein the sleeve is made of a layer of a compliant biocompatible material.

11. The device of claim 1, wherein the sleeve is elastically deformable in the opening of the operating site of the eye to match a shape or contour of the opening.

12. The device of claim 1, further comprising means for irrigating fluid or aspirating fluid, tissue or other debris through a space between the shaft and the sleeve, and through the port into or out of the eye.

13. The device of claim 12, wherein the means are attached to the device.

14. The device of claim 1, wherein the sleeve is translucent to allow further visualization of an anterior chamber and confirmation of fluid transfer within the sleeve.
